(19) United States
(12) Plant Patent Application Publication
(10) Pub. No.: US 2005/0210553 P1
(43) Pub. Date: Sep. 22, 2005

(54) PRUNUS ROOTSTOCK NAMED 'AP-1'

(50) PRUNUS CERASIFERA X PRUNUS PERSICA; PRUNUS ROOTSTOCK

(75) Inventor: Guennadiy Eremin, Krasnodar (RU)

Correspondence Address: THE WEBB LAW FIRM, P.C., 700 KOPPERS BUILDING, 436 SEVENTH AVENUE, PITTSBURGH, PA, 15219, US

(73) Assignee: Varieties International, LLC, Lafayette, OR (US)

(21) Appl. No.: 10/802,611

(22) Filed: Mar. 17, 2004

(51) Int. Cl. A01H 5/00

(52) U.S. Cl. PLT/180

(57) ABSTRACT

A new and distinct Prunus cerasifera×Prunus persica plant used as a rootstock for many different varieties that exhibits desirable propagation characteristics and pink flowers.

### BOTANICAL CLASSIFICATION

Prunus cerasifera×Prunus persica

### VARIETAL DENOMINATION

‘AP-1’

### BACKGROUND OF THE INVENTION

The present invention comprises a new and distinct cultivar of Prunus cerasifera×Prunus persica used as a rootstock known by the varietal name ‘AP-1’. The new variety was discovered in the Krasnodar region of Russia around 1986. The new variety is the result of planned breeding between a Prunus cerasifera plant (female parent) and a Prunus persica plant (male parent). The new variety differs from its parents in that its flowers are big and pink, it is not fruit bearing; ‘AP-1’ has wide elliptical leaves, and it has serrated leaf margins. The purpose of breeding ‘AP-1’ was to provide a productive clonal rootstock for peach and plum varieties. The new variety has been trial and field tested and has been found to retain its distinctive characteristics and remain true to type through successive propagations. The following characteristics distinguish ‘AP-1’ from other varieties known to the breeder:

1. ‘AP-1’ may be propagated via hardwood or softwood cuttings;

2. ‘AP-1’ has a strong root system;

3. ‘AP-1’ is resistant to nematodes, chlorosis, Cytospora, and Verticillium; and

4. ‘AP-1’ is drought resistant.

### DESCRIPTION OF THE DRAWINGS

The accompanying photographic drawings illustrate the new variety, with the color being as nearly true as is possible with color illustrations of this type:

FIG. 1 shows the new variety grown to a flowering plant;

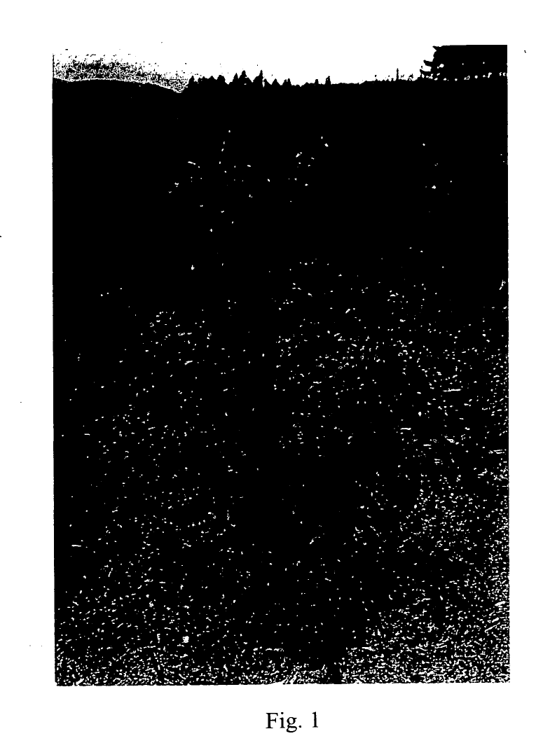

FIG. 2 shows a close-up view of leaves of the new variety; and

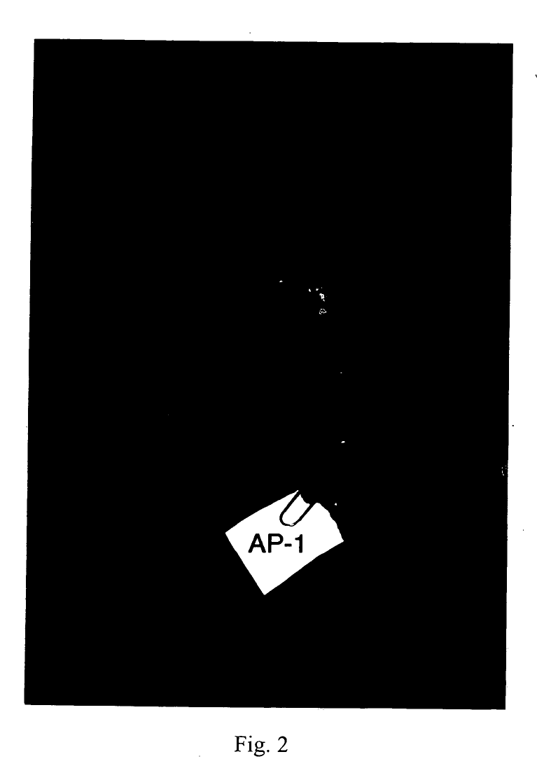

FIG. 3 illustrates the roots and branches of the new variety.

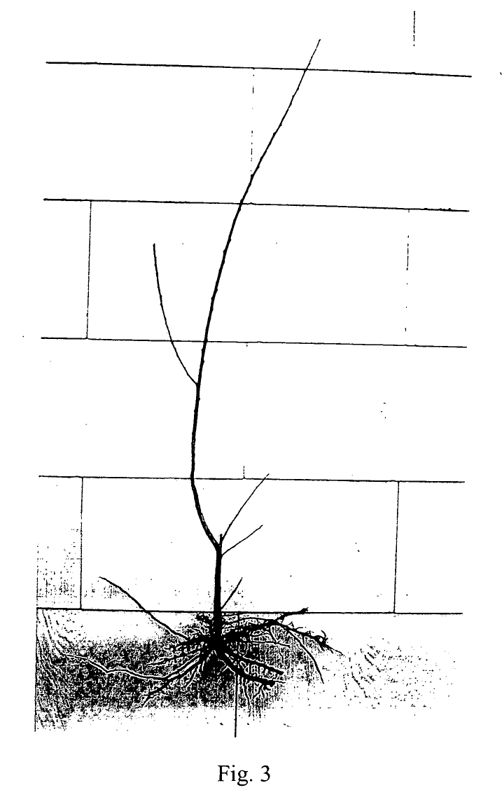

### DETAILED BOTANICAL DESCRIPTION

The following detailed description sets forth the characteristics of the new cultivar. The data which defines these characteristics were collected by asexual reproductions via cuttings carried out in the Krasnodar Region of Russia. The new variety was grown under warm, dry Summer conditions with a temperature range of 70 to 105° F. The Winter months are mild with lows to −10° F. The Spring and Fall months are humid. The color readings were taken in natural daylight.

### Tree

Use: Rootstock.
Fruit bearing: No fruit observed to date on ‘AP-1’.
Size (generally): Medium.
   Height at 2 years.—3 feet.
   Spread at 2 years.—1.5 feet.
Form: Rounded; spreading.
Trunk:
   Size.—Medium.
   Bark color.—177A.
   Surface texture.—Smooth or with very weak pubescence.
   Diameter.—9.0 mm at 3 years when grown in a pot.
   Texture.—Smooth.
   Lenticel number.—7-12 per cm at 3 years.
Branches:
   Surface texture.—Smooth.
   Young bark color.—178B.
   Mature bark color.—179A.
   Internode length.—2.0-2.5 cm.
   Lenticel size.—3.0 mm×1.0 mm.
   Lenticel number.—Few; none observed on a 1 year old branch.
   Lenticel shape.—Oval.
   Bud shape.—Conical.
   Bud size.—3.0 mm.
   Bud texture.—Pubescent.
   Diameter.—5.0 mm at 1 year.
   Crotch angle.—60°.
Leaves:
   Bud.—Length: 1.5 mm. Diameter: 1.5 mm. Color: 171A.
   Length.—14.0 cm.
   Width.—5.0 cm.
   Surface texture.—Upper surface is glossy.
   Form.—Narrow; elliptic.
   Color.—Lower surface: Light green. Upper surface: Green.
   Mid-vein.—Size: Thin. Color: White, with the center being light pink.
   Petiole.—Length: 1.0 cm. Thickness: 1.5-2.0 mm. Color: Pink.
   Leaf glands.—Absent.
   Stipules.—Size: Up to 2.0 cm. Color: Green.
Flowers:
   Bloom time.—Flowers reach full bloom the third week of March at the same time as plum trees and ahead of peach trees.
   Size.—35.0-38.0 mm.
   Color.—Light pink.
   Number.—1 to 3 flowers per flower bud.
   Fragrance.—None.
   Petals.—Number: 5 per flower. Shape: Clam-shaped. Length: 20.0 mm. Width: Widest point is 18.0 mm; the base and apex are both 15.0 mm. Texture: Smooth. Color: 62D. Appearance: Petals overlap at full bloom.
Sepals:
   Number.—5 per bloom.
   Shape.—Ovoid to round.
   Length.—4.0 mm.
   Width.—3.0 mm.
   Color.—138C in the middle.
Reproductive organs:
   Anthers.—Number: 34 to 40. Color: 17B. Filament length: 13.0 mm. Filament color: 62C.
   Stigma.—Number: 1. Position: Below the anthers.
   Style.—Length: 4.0 mm. Color: 130D.
Fruit: None.

### Soil Adaptation and Tolerance

Chlorosis: ‘AP-1’ is resistant to high pH and lime which causes lime-induced chlorosis.
Wet soil: Resilient.
Cold temperatures: Hardy to Zone 4.
Asphixia: Resistant; will survive water-logged soil for extended time periods.

### Multiplication Ability

Layering: None.
Hardwood cuttings: Great propagation.
Softwood cuttings: Great propagation.

### Pathogen Resistance

Fungal disease: Resistant to all fungi exposed to the new variety.
Insects: Not resistant.
Mites: Weak resistance.
Viruses: Resistant.
Diseases: Resistant to Cytospora, Verticillium, and bacterial canker.
Root-knot nematodes: Immune, according to three tests in Spain.
Lesion nematodes: Highly resistant.

### Performance as a Rootstock When Grafted

Root sprouts (suckering): Suckers (shoots that grow from the rootstock and disrupt the growth of the scion) are not present.
Anchorage: Very good.
Compatibility: Good compatibility with all varieties of peaches, almonds, nectarines, Japanese plums, Russian plums, and apricots known to the breeder.
Vigor: Medium to strong.

1. A new and distinct variety of Prunus cerasifera×Prunus persica plant, as illustrated and described.
